(19) United States
(12) Patent Application Publication
(10) Pub. No.: US 2025/0259720 A1
(43) Pub. Date: Aug. 14, 2025

(54) METHOD FOR MANAGING MEDICAL DATA

(71) Applicant: Roche Diabetes Care, Inc., Indianapolis, IN (US)

(72) Inventor: Matthias Gieselmann, Wien (AT)

(21) Appl. No.: 18/758,236

(22) Filed: Jun. 28, 2024

Related U.S. Application Data
(63) Continuation of application No. PCT/EP2022/087945, filed on Dec. 28, 2022.

(30) Foreign Application Priority Data
Dec. 31, 2021 (EP) 21 218 500.3

(51) Int. Cl. G16H 10/60

(52) U.S. Cl. CPC G16H 10/60

(57) ABSTRACT

This disclosure relates to a method, a mobile device, and a computer program, for managing medical data such as, e.g., glucose concentrations, by an electronic disease management system. In the method a change of the local time between generation of a first set of medical data and a second set of medical data is detected.

### RELATED APPLICATIONS

This application is a continuation of International Application Serial No. PCT/EP2022/087945, filed Dec. 28, 2022, which claims priority to European Patent Application Serial No. 21 218 500.3, filed Dec. 31, 2021, the entire disclosures of both of which are hereby incorporated herein by reference.

### BACKGROUND

The disclosure relates to a method, a mobile device, and a computer program for managing medical data such as, e.g., glucose concentrations, by an electronic disease management system comprising detecting a change of the local time between generation of a first set of medical data and a second set of medical data.

Diabetes is a disease in which the body's ability to produce or respond to the hormone insulin is impaired, resulting in abnormal metabolism of carbohydrates and elevated levels of glucose in the blood. If left untreated, diabetes can cause many complications. Acute complications can include diabetic ketoacidosis, hyperosmolar hyperglycemic state, or death. Serious long-term complications include cardiovascular disease, stroke, chronic kidney disease, foot ulcers, and damage to the eyes.

Self-monitoring of glucose concentrations with glucose meters or continuous glucose monitoring sensors and the self-administration of insulin with pens or pumps is the typical method of persons with diabetes for treating their diabetes.

Persons with diabetes may further use a computing device such as a portable communication device (e.g., a smart phone, a personal digital assistant, a tablet or similar device) connected to their medical device(s). The results of a blood glucose measurement or the units of delivered insulin are, e.g., wirelessly transmitted from the medical device to the computing device.

The computing device may process, store, analyze and display the medical data in a graph over time. The diabetes-related medical devices, such as blood glucose meters, continuous glucose monitoring sensors, insulin pens or pumps therefore include a means for tracking time, such as an internal digital clock, and can store the time-stamped medical data (e.g., measured glucose concentrations, delivered insulin units) for real-time or later transmission to the computing device. A cumulated quantity of insulin delivered to the user, the so-called insulin on board, may also be viewed or a glucose trend may be determined based on the imported data including the time stamps. In any case, the reliability and comprehensibility of the data with the corresponding time stamps is essential for the further analysis and for treatment decisions of the patient.

Methods for preparing time related data from different medical devices are known in the art. For example, WO 2016/087714 A1 discloses a method for monitoring of a glucose level of a user. A server communicates with blood glucose meters over internet. The blood glucose meters send their own current time along with measurement values and time stamps. The server then compares the current time sent by the glucose meter with a server time, detect a possible discrepancy and adjusts the timestamps to a known and correct reference time, such as the Universal Coordinated Time (UTC).

EP 2 988 672 B1 discloses a diabetes management system comprising a plurality of blood glucose meters in data communication with a diabetes management application residing on a mobile phone. Each meter has its internal real time clock (RTC) and the mobile phone has its internal phone time, which is set relative to GMT if the mobile phone is connected to a network. When the mobile phone is not connected to a network, the phone time is set to be equal to an RTC time of a meter. For each meter the diabetes management application determines a delta time between the phone time and the RTC of the meter. The management application stores received measurement values with RTC time stamps from the meter along with the corresponding computed time delta. Consequently, the management application computes a new time stamp for each measuring value by adding the time delta to the corresponding RTC time stamp. The measuring values are stored along with the computed new time stamp.

As long as all medical data are gained in one time zone and all times and clocks in the medical devices and in the computing device are synchronized to a common time the processing of the imported data in the computing device works fine. However, problems arise if medical data collected in different time zones shall be displayed and analyzed because in this case some of the time stamps may relate to the time prior to the time change while other time stamps may relate to the time after the time change.

WO 2019/199920 discloses that a person with diabetes (PWD) may often travel between locations in different time zones. As such, the user interface of a mobile device can utilize information on the universal concept of time and the current time zone of the mobile device to display time related treatment information to the patient with diabetes based on the current (new) time zone of the mobile device. The mobile device may, e.g., detect the current time zone for the mobile device and convert the time stamp associated with a previously recorded bolus dosage information from the universal concept of time to a time based on the current time zone for the mobile device.

WO 2019/243907 discloses an app detecting time change events and adding delta time to time stamps generated with the previous time of a blood glucose meter (BMG). This is repeated for all time stamps of a current import and for all time changes of the BGM time that may affect the imported time stamps. Finally, the time difference between the app UTC time and the latest BGM time is added to the BGM time stamps. This yields to the new and additional UTC time stamp. The converted UTC time stamps are again converted to a display time stamp by adding a time difference between the current local app time and the current app UTC time.

Despite the advances made regarding consideration of time changes, managing medical data created in different time zones remains a challenge. Specifically, display and usability of displayed medical data needs to be enhanced. When the time zone changes and the displayed time of past events and values is changed, this can, e.g., mean that therapy events that happened during the afternoon before a time zone change now show up during the night. This makes it harder to retrospectively relate therapy events.

### SUMMARY

This disclosure teaches methods and devices which address the above mentioned technical challenges of managing medical data using mobile devices such as consumer-electronics mobile devices. Specifically, methods and devices are disclosed which provide an easy way for tracking or interpreting past medical data which may also help for determining an appropriate current or future treatment for managing a medical condition (status), such as a diabetes condition.

As used in the following, the terms “have,” “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B,” “A comprises B” and “A includes B” may both refer to a situation in which, besides B, no other element is present in A (i.e., a situation in which A solely and exclusively consists of B) and to a situation in which, besides B, one or more further elements are present in entity A, such as element C, elements C and D or even further elements.

Further, it shall be noted that the terms “at least one,” “one or more” or similar expressions indicating that a feature or element may be present once or more than once typically will be used only once when introducing the respective feature or element. In the following, in most cases, when referring to the respective feature or element, the expressions “at least one” or “one or more” will not be repeated, non-withstanding the fact that the respective feature or element may be present once or more than once. It shall also be understood for purposes of this disclosure and appended claims that, regardless of whether the phrases “one or more” or “at least one” precede an element or feature appearing in this disclosure or claims, such element or feature shall not receive a singular interpretation unless it is made explicit herein. By way of non-limiting example, the terms “medical data,” “time stamp,” and “graph,” to name just a few, should be interpreted wherever they appear in this disclosure and claims to mean “at least one” or “one or more” regardless of whether they are introduced with the expressions “at least one” or “one or more.” All other terms used herein should be similarly interpreted unless it is made explicit that a singular interpretation is intended.

Further, as used in the following, the terms “preferably,” “more preferably,” “particularly,” “more particularly,” “specifically,” “more specifically” or similar terms are used in conjunction with optional features, without restricting alternative possibilities. Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding alternative embodiments of the invention, without any restrictions regarding the scope of the invention and without any restriction regarding the possibility of combining the features introduced in such way with other optional or non-optional features of the invention.

In a first aspect, a computer implemented method for managing medical data by an electronic disease management system comprising a processing unit and a display unit is disclosed. The method comprises the following steps (it shall be noted that the method may comprise further method steps which are not listed below):
a) receiving by the processing unit a first set of a patient's medical data wherein the medical data of the first set of medical data are associated with respective first time stamps indicating the respective times of generation of the medical data of the first set of medical data according to a first local time,
b) associating by the processing unit the medical data of the first set of medical data with respective second time stamps indicating the respective times of generation of the medical data of the first set of medical data according to a reference concept of time,
c) receiving by the processing unit a second set of the patient's medical data wherein the medical data of the second set of medical data are associated with respective third time stamps indicating the respective times of generation of the medical data of the second set of medical data according to a second local time,
d) associating by the processing unit the medical data of the second set of medical data with respective fourth time stamps indicating the respective times of generation the medical data of the second set of medical data according to the reference concept of time,
e) detecting by the processing unit a change the local time between generation of the first set of medical data and the second set of medical data,
f) providing first display signals by the processing unit representing the first set of medical data and the second set of medical data in a first graph over time wherein the medical data of the first set of medical data and the second set of medical data are in chronological order of their times of generation according to the reference concept of time in the first graph and the spacing between medical data in the direction of time in the first graph is directly proportional to the difference in their times of generation according to the reference concept of time, and the change of the local time between generation of the first set of medical data and the second set of medical data is indicated in the first graph.

The term “medical data” as used herein is a broad term and is to be given its ordinary and customary meaning to a person of ordinary skill in the art and is not to be limited to a special or customized meaning. The term specifically may refer, without limitation, to health-related information that is associated with a patient. The medical data, as an example, may comprise quantitative medical data, such as concentrations of one or more analytes in a bodily fluid of a patient, drug doses administered to a patient, and/or amounts of carbohydrates ingested by a patient. The term medical data may thus comprise one or more types of medical data, such as, e.g., glucose concentrations in a bodily fluid of the patient, hormone (e.g., insulin, c-peptide and/or glucagon) concentrations in a bodily fluid of the patient, insulin doses administered to the patient, and/or carbohydrate amounts ingested by the patient. In one example, the analyte is glucose. The term medical data may particularly comprise a patient's glucose concentrations in a bodily fluid, e.g., blood glucose concentrations, glucose concentrations in interstitial fluid, urine, saliva or another type of body fluid, and/or insulin doses administered to the patient. In one embodiment the medical data comprise a patient's blood glucose concentrations and/or administered insulin doses.

In one example, the medical data of the first set of medical data are analyte concentrations and/or medication doses, and the medical data of the second set of medical data are further analyte concentrations and/or further medication doses. In another example, the medical data of the first set of medical data are glucose concentrations, and the medical data of the second set of medical data are further glucose concentrations.

A set of medical data comprises one or more medical data. For example, a first set of medical data comprises at least 3 medical data (m1, m2, m3) of patient and a second set of medical data comprises at least 3 further medical data (m4, m5, m6) of the patient. The medical data of the first and/or the second set of medical data may all relate to the same type of medical data for a patient (e.g., all blood glucose concentrations) or they may relate to different types of medical data for a patient (e.g., some of the medical data of the first and/or the second set of medical data are blood glucose concentrations of the patient while other medical data of the first and/or the second set of medical data are insulin doses administered to the patient). In one embodiment, the medical data of the first and second set of medical data are of the same type, such as glucose concentrations in a patient's bodily fluid (e.g., blood or interstitial fluid) or insulin doses administered to the patient.

The term “electronic disease management system” as used herein is a broad term and is to be given its ordinary and customary meaning to a person of ordinary skill in the art and is not to be limited to a special or customized meaning. The term electronic disease management system (in short: system herein) specifically may refer, without limitation, to a mobile communication device such as a mobile phone, in particular a smart phone. Additionally or alternatively, the system may refer to personal computer, a tablet computer or another type of computer. The system comprises a processing unit. The system may also comprise a memory. The memory may, e.g., store previously received medical data and associated time stamps. The system further comprises a display unit.

The processing unit is configured to receive medical data and associated respective first and third time stamps and to further associate the received medical data with respective second time stamps. The processing unit is further configured to provide display. The processing unit is, e.g., configured to provide display signals to the display unit comprising a touch sensitive display. Additionally or alternatively the display unit may comprise a liquid crystal display, thin film transistor display, light emitting diode display, or any other device capable of transforming signals from a processor into an optical output.

Medical data and associated respective first and third time stamps may, e.g., be received from one or more medical devices, such as a glucose meter and/or a medication (e.g., insulin) delivery device. Transmission of the medical data and the associated first and third time stamps may be by wired transfer, or particularly by wireless transfer (e.g., using Bluetooth or NFC technology). Medical data and associated respective first and third time stamps may additionally or alternatively be received from the memory of the system and/or from an alternative data source, such as a cloud source.

The processing unit may be provided with one or more processors. The one or more processors may be integral with a single component of the system. However, it is noted that the one or more processors may also be separately located within discrete components such as, for example, a glucose meter, a medication delivery device, and a mobile phone, wherein the glucose meter and the medication delivery device may be communicatively coupled to the mobile phone. In this case the mobile phone, as well as the glucose meter and the medication delivery device may be all be considered components of the system. Accordingly, the system may include a plurality of components each having one or more processors that are communicatively coupled with one or more of the other components.

The method of this disclosure comprises:
a) receiving by the processing unit a first set of a patient's medical data wherein the medical data of the first set of medical data are associated with respective first time stamps indicating the respective times of generation of the medical data of the first set of medical data according to a first local time.

The term “local time” (also referred to as “time zone”) as used herein is a broad term and is to be given its ordinary and customary meaning to a person of ordinary skill in the art and is not to be limited to a special or customized meaning. The term local time (or time zone) specifically may refer, without limitation, to a time that is specific to a geographic location (or zone defined due to regional needs like boundaries between countries and their subdivisions) and that has a specified time offset to the Coordinated Universal Time. The specified time offset may depend on the date when considering time changes due to the daylight saving time. A local time's offset to Coordinated Universal Time (which is commonly referred to as the UTC) is usually a whole number of hours, but a few local times are offset by an additional 30 or 45 minutes, such as in India and Nepal. The first local time is, e.g., Central European Time (CET), which is 1 hour ahead of UTC; Central European Summer Time (CEST), which is 2 hours ahead of UTC; Eastern Time (ET), which is 5 hours behind UTC; Eastern Daylight Time (EDT), which is 4 hours behind UTC; Pacific Time (PT), which is 8 hours behind UTC; Pacific Daylight Time (PDT), which is 7 hours behind UTC, or China Standard Time (CST), which is 8 hours ahead of UTC. The second local time is, e.g., Central European Time (CET), which is 1 hour ahead of UTC; Central European Summer Time (CEST), which is 2 hours ahead of UTC; Eastern Time (ET), which is 5 hours behind UTC; Eastern Daylight Time (EDT), which is 4 hours behind UTC; Pacific Time (PT), which is 8 hours behind UTC; Pacific Daylight Time (PDT), which is 7 hours behind UTC, or China Standard Time (CST), which is 8 hours ahead of UTC, provided that the second local time is different from the first local time in this disclosure.

The Coordinated Universal Time is independent of geographic location. UTC is thus also independent of the location where a patient is when generating medical data or where any component of the system is located. In this disclosure the first local time is different from the second local time, i.e., the time offset of the first local time from the Coordinated Universal Time is different from the time offset of the second local time from the Coordinated Universal Time.

Generating medical data, e.g., refers to the creation of the medical data, e.g., the measuring of concentrations of an analyte in bodily fluid of a patient or the delivering of amounts of a drug to a patient. Time of generation of the medical data of the first set of medical data according to the first local time refers to the respective current first local times when the medical data were generated, e.g., the current first local times when the analyte concentrations of the patient were measured or when the amounts of drug were delivered to the patient. Time of generation of the medical data of the second set of medical data according to the second local time refers to the respective current second local times when the medical data were generated, e.g., the current second local times when the analyte concentrations of the patient were measured or when the amounts of drug were delivered to the patient. Indication of the respective times of generation of the medical data according to a local time includes an indication that enables a conversion of the time stamp to the reference concept of time by the processing unit. The time stamps indicating respective times of generation of medical data according to a local time thus, e.g., comprise information on i) current time of day (e.g., 14:35), ii) current date (e.g., 11 Apr. 2021) and iii) local time (e.g., CET) or time-offset of the local time to UTC (e.g., UTC+01:00) to indicate unequivocally the respective times of generation of the medical data.

Medical data that are associated with time stamps indicating the respective times of generation according to a first local time, e.g., refers to glucose concentrations in a bodily fluid of the patient that were measured in a first time zone having a first local time or amounts of drug doses delivered to the patient in a first time zone having a first local time. When a patient is located in a first time zone having a first local time he, e.g., generates a first set of glucose concentrations in bodily fluid by conducting corresponding glucose concentration measurements in the first time zone having the first local time and/or administered drug doses by conducting corresponding drug dose self administrations in the first time zone having the first local time. The glucose concentration measurements may be conducted by the patient being in the first time zone having the first local time using a glucose meter, such as a blood glucose meter or a continuous glucose measurement device. The drug (e.g., insulin) dose administrations may be conducted by the patient being in the first time zone having the first local time using a drug delivery device, e.g., an insulin pen or an insulin pump. When conducting a glucose measurement with a glucose measurement device or when delivering an amount of insulin with an insulin delivery device (e.g., an insulin pen or an insulin pump), the medical device may save not only the measured glucose concentration or the delivered amount of insulin, but may also store the time of generation of the medical data as a time stamp (associated time stamp indicating the respective time of generation of the medical data according to a first local time); in this example the time of the respective glucose concentration measurement or the time of the respective delivery of the amount of insulin) therewith indicating the current local time when the measurement took place or when the drug delivery took place according to the first local time. In this disclosure, the processing unit not only receives the first set of medical data, e.g., a plurality of glucose concentrations measured in a bodily fluid, but the medical data of the first set of medical data are associated with respective time stamps indicating the respective times of generation of the medical data of the first set of medical data according to the first local time. The processing unit, besides receiving the first set of medical data, thus also receives the respective time stamps indicating the respective times of generation of the medical data of the first set of medical data according to the first local time. In one embodiment, association of the medical data with respective first time stamps indicating the respective times of generation of the medical data (116) of the first set of medical data according to a first local time may not happen on the medical device, but may happen on a smart phone to which the medical data are wirelessly transmitted. The medical data with associated respective time stamps indicating the respective times of generation of the medical data according to a local time may be saved in a memory of the system from which the processing unit of the system can also receive the medical data with the associated respective time stamps (e.g., instead of receiving them from a medical device).

The method of this disclosure further comprises:
b) associating by the processing unit the medical data of the first set of medical data with respective second time stamps indicating the respective times of generation of the medical data of the first set of medical data according to a reference concept of time.

The term “reference concept of time” as used herein is a broad term and is to be given its ordinary and customary meaning to a person of ordinary skill in the art and is not to be limited to a special or customized meaning. The term reference concept of time specifically may refer, without limitation, to a concept of time that is independent of the time zone in which the system is located. The reference concept of time is, e.g., the Coordinated Universal Time (UTC). For example, the reference concept of time may alternatively be Unix Time, or any other time that is not dependent on a particular time zone. For associating the medical data of the first set of medical data with respective second time stamps the processing unit can convert the times of generation (also referred to as generation times) according to the first local time into times of generation according to the reference concept of time wherein the generation of the converted time stamps (second time stamps) comprises the step of adding a first delta to the times of generation according the first local time wherein the first delta corresponds to the time offset (delta) of the first local time to the reference concept of time. The time offset may be a positive or negative value. That means the term “adding” comprises an addition of a positive value to the time stamp as well as a subtraction of a value from the time stamp.

The method of this disclosure further comprises:
c) receiving by the processing unit a second set of the patient's medical data wherein the medical data of the second set of medical data are associated with respective third time stamps indicating the respective times of generation of the medical data of the second set of medical data according to a second local time, and
d) associating by the processing unit the medical data of the second set of medical data with respective fourth time stamps indicating the respective times of generation of the medical data of the second set of medical data according to the reference concept of time.

Reference is made to the description above which is transferable to steps c) and d). Instead of a first set of medical data a second set of medical data is received in step c) and the third time stamps indicate the respective times of generation according to the second local time (whereas the first time stamps indicated the respective times of generation according to the first local time). The second set of medical data is then associated with respective fourth time stamps (whereas the first set of medical data were associated with respective second time stamps) indicating the respective times of generation of the medical data of the second set of medical data according to the reference concept of time. By associating the medical data of the first and second set of medical data with respective time stamps indicating the respective times of generation according to the reference concept of time, they can be set in relation to each other respecting the chronology of generation. In one embodiment the second set of medical data is generated after the first set of medical is generated and the patient travelled from a first time zone having the first local time to a second time zone having the second local time between generation of the first set of medical data and the second set of medical data, wherein the first set of medical data were generated in the first time zone and the second set of medical data were generated in the second time zone. In one example, the second set of medical data is generated and received after the first set of medical data is generated and received by the processing unit.

The method further comprises:
e) detecting by the processing unit a change of the local time between generation of the first set of medical data and the second set of medical data.
f) providing first display signals by the processing unit representing the first set of medical data and the second set of medical data in a first graph over time wherein the medical data of the first set of medical data and the second set of medical data are in chronological order of their times of generation according to the reference concept of time in the first graph and the spacing between medical data in the direction of time in the first graph is directly proportional to the difference in their times of generation according to the reference concept of time, and the change of the local time between generation of the first set of medical data and the second set of medical data is indicated in the first graph.

The change of the local time can, e.g., be detected by the processing unit in the change of the time-offset of the local time to UTC indicated by the first and third time stamps. The change of the local time can, e.g., also be detected by the processing unit in a change of location data (e.g., detectable by GPS signals received by the processing unit) associated with the first and second set of medical data. The processor may link location data with time zone information. In one embodiment a change of the local time is, e.g., detected when the first local time is CET and the second local time is GMT.

The first graph may, e.g., be a line graph or a scatter plot to show the change of the medical data over time. The first graph may, e.g., visualize glucose concentrations over time in a line graph or a scatter plot. Time may be on the x-axis in the graph and, e.g., glucose concentration may be on the y-axis in the graph. The axes themselves do not necessarily need to be displayed in the graph. The first graph comprises the first set of medical data and the second set of medical data and an indication of the change of the local time between generation of the first set of medical data and the second set of medical data.

The term “chronological order” as used herein is a broad term and is to be given its ordinary and customary meaning to a person of ordinary skill in the art and is not to be limited to a special or customized meaning. The term chronological order specifically may refer, without limitation, to the arrangement of the medical data in the graph in the order of their generation, wherein the order of their generation is determined according to the reference concept of time. Due to the second and fourth time stamps relying on the same reference concept of time arrangement of the medical data in chronological order is easily possible.

The spacing between medical data in the direction of time in the first graph being directly proportional to the difference in their times of generation according to the reference concept of time means that the distance in spacing (ΔX) in the direction of time in the graph of two medical data m1 and m2 with times of generation t1 and t2 (wherein t2>t1) according to the reference concept of time follows the equation ΔX=A*ΔT, wherein A is a constant and ΔT is the difference of the times of generation of the two medical data according to the reference concept. This allows for an easy interpretation of the medical data by the patient. Medical data generated with equal time difference according to the reference concept of time are equally spaced apart in the direction of time in the first graph. Assuming as an example a first set of medical data m1, m2 and m3 generated in this order and a second set of medical data m4, m5 and m6 generated in this order and after m1-m3 have been generated, and assuming that m1 and m2 have been generated with a time difference of 1 hour according to the reference concept of time, that m3 and m4 have been generated with a time difference of 1 hour according to the reference concept of time, and that m5 and m6 have been generated with a time difference of 1 hour according to the reference concept of time, m1-m6 can be plotted in a line graph or scatter plot, wherein m1, m2, m3, m4, m5, and m6 are shown in this order in the graph in the direction of time, and m1 and m2, m3 and m4, and m5 and m6 are equally spaced apart in the direction of time in the graph. The change of the local time between generation of the first set of medical data and the second set of medical data is indicated in the first graph. In the provided example it will thus be visible between m3 and m4. The way of representation in the first graph further has the advantage that events such as glucose measurements are visualized in the order and with absolute time differences as they were experienced by the patient, no data is lost, and the change of the local time is visible to the patient simplifying an interpretation of the data for the patient.

In one example, the method comprises displaying the entire first graph on the display unit. The patient can thus view the entire first graph at the same time. In another example the method comprises displaying only a partial area of the first graph. The patient can thus view only a partial area of the first graph at a time. But, in case of a touch sensitive display unit, the patient can scroll through the first graph to view all parts of the first graph. The processing unit may thus provide first display signals by the processing unit to the display unit which must not necessarily display all first display signals at the same time, but a first partial area of the first graph may be displayed at a first time, and a second partial area of the first graph may be displayed at a second time, e.g., when the patient scrolls through the graph on a touch sensitive display. In another embodiment, all first display signals are displayed on the display unit for the patient to view them at the same time. In this case, the patient, in case of a touch sensitive display, thus does not need to scroll through the graph to view all medical data of the first and second set of medical data. In one example the first set of medical data and the second set of medical are generated within an absolute time frame of 24 or 18 hours or less. In this example, the entire first graph may be displayed on the display unit for the patient to view at the same time.

In one embodiment, the method comprises displaying a target zone for the medical data in the first graph. The target zone indicates boundaries between which the quantitative medical data should fall. Outliers (medical data that fall outside the boundaries) indicate that the therapy for treating the patient's disease should be adjusted.

The method may further comprise:
b1) providing second display signals by the processing unit representing the first set of medical data in a second graph over time wherein the medical data of the first set of medical data are in chronological order of their times of generation according to the first or reference concept of time in the second graph and the spacing between medical data in the direction of time in the second graph is directly proportional to the difference in their times of generation according to the reference concept of time.

In one example, the method comprises displaying the entire second graph on the display unit. The patient can thus view the entire second graph at the same time. In another example the method comprises displaying only a partial area of the second graph. The patient can thus view only a partial area of the second graph at a time. In case of a touch sensitive display unit, the patient can scroll through the second graph to view all parts of the graph. The processing unit may thus provide second display signals by the processing unit to the display unit which must not necessarily display all second display signals at the same time, but a first partial area of the second graph may be displayed at a first time, and a second partial area of the second graph may be displayed at a second time, e.g., when the patient scrolls through the graph on a touch sensitive display. In another embodiment, all second display signals are displayed on the display unit for the patient to view them at the same time. In this case, the patient, in case of a touch sensitive display, thus does not need to scroll through the graph to view all medical data of the first set of medical data.

The second display signals may be provided to the display unit (and displayed on the display unit) before the second set of medical data is received by the processing unit. In one embodiment, when the first graph or a partial area thereof are displayed on the display unit, the second graph or a partial area thereof are no longer displayed on the display unit. In one embodiment, the first display signals replace the second display signals.

The first set of medical data may be generated while the patient is in a first time zone having the first local time; the second set of medical data may be generated while the patient is in a second time zone having the second local time. The second display signals may be provided to the display unit when the patient is in the first time zone, and the first display signals may be provided to the display unit when the patient is in the second time zone.

In one embodiment, the second display signals comprise signals for representing a time scale in the second graph which indicates when the medical data of the first set of medical data were generated according to the first local time. The time scale may particularly be shown on the x-axis of the second graph. The time scale, e.g., comprises at least two time scale tick marks and at least two time scale tick mark time labels so that the patient can identify based thereon the respective times of generation of the first set of medical data according to the first local time when looking at the second graph.

Additionally or alternatively, the first display signals, e.g., comprise signals for representing a first time scale part in the first graph which indicates when the medical data of the first set of medical data were generated according to the first local time, and a second time scale part in the first graph which indicates when the medical data of the second set of medical data were generated according to the second local time.

The first and second time scale parts may particularly be shown on the x-axis of the first graph. The first time scale part, e.g., comprises at least two first tick marks and at least two first tick mark time labels so that the patient can identify based thereon the respective times of generation of the first set of medical data according to the first local time when looking at the first graph. The second linear time scale part, e.g., comprises at least two second tick marks and at least two second tick mark time labels so that the patient can identify based thereon the respective times of generation of the second set of medical data according to the second local time when looking at the first graph.

In one embodiment, all tick marks are evenly spaced in the first graph. Specifically, the distance between two neighboring tick marks shown in the first graph may be the same (no matter whether the respective neighboring tick marks are two first tick marks, two second tick marks or a first and a second tick mark). The at least two first tick mark labels may indicate linear ascending time of day according to the first local time. The at least two second tick mark labels may indicate linear ascending time of day according to the second local time. However, due to the change of the local time between generation of the first set of medical data and the second set of medical data, there may appear to be a non-linearity between the last of the first tick mark labels and the first of the second tick mark labels. In one embodiment (e.g., when travelling from east to west) the first one of the second tick mark labels may indicate a time of day that is the same or before the time of day of the last one of the first tick mark labels. In one embodiment (e.g., when travelling from west to east) the first one of the second tick mark labels may indicate a time of day that is later than a time of day that would result from using purely linear ascending tick mark labels.

As, e.g., insulin secretion underlies a circadian rhythm it is important for the patient (or the health care practitioner) to view not only the patient's glucose concentrations, but also the respective times of generation according to the local time where the data were generated in a graph. If all past times of generation of medical data are converted to times of generation according to the current local time of where a patient is located and the medical data are displayed in a graph over time indicating for all medical data only the current local time, patients (or the health care practitioner) may misinterpret, e.g., insulin needs at specific times of the day or overnight.

In one embodiment, the display unit is a touch sensitive display unit comprising a controller unit to control the display unit, and optionally a sensing unit. The sensing unit, e.g., senses touch gestures on the display. The controller is, e.g., adapted to receive the first display signals and to control the display unit to display the first graph or a partial area thereof at a time. The controller may also be adapted to receive the second set of display signals and to control the display unit to display the second graph or a partial area thereof at a time prior to receiving the second set of medical data.

The controller is, e.g., adapted to receive the first display signals and to control the display unit to display a first partial area of the first graph at a first time, wherein the sensing unit is adapted to sense a first scroll through action of the user in backward or forward direction of the displayed chronologically ordered medical data in the first partial area of the first graph, and the controller in response to the sensing of the first scroll through action is adapted to control the display unit to display a second partial area instead of the first partial area of the first graph at a second time, the second partial area of the first graph lying at least partially backward of forward of the previously displayed chronologically ordered medical data in the first partial area of the first graph. When time is on the x-axis of the graph the backward scroll through action may be a swiping gesture to the right with a finger on the display. When time is on the x-axis of the graph the forward scroll through action may be a swiping gesture to the left with a finger on the display.

In another example, the controller is, e.g., adapted to receive the first display signals and to control the display unit to display a first partial area of the first graph at a first time, wherein the sensing unit is adapted to sense a first zoom out action of the user in the first partial area of the first graph, and the controller in response to the sensing of the zoom out action is adapted to control the display unit to display a second partial area of the first graph at a second time, the second partial area of the first graph comprising the first partial area and further medical data lying backward of forward of the previously displayed chronologically ordered medical data in the first partial area of the first graph.

In one embodiment, the first display signals comprise additional signals representing the first set of medical data and the second set of medical data in a list in chronological order of their times of generation according to the reference concept of time, wherein next to each medical data of the first set of medical data the respective time of generation according to the first local time is indicated and next to each medical data of the second set of medical data the respective time of generation according to the second local time is indicated. The list (also termed first list herein) or a part thereof may be displayed on the display unit in addition to displaying the first graph or a partial area of the first graph at a first time. The controller may further be adapted to receive the first display signals and to control the display unit to display a first part of the list on the display unit of the user's electronic device at the first time, wherein the sensing unit is adapted to sense a second scroll through action of the user in backward or forward direction of the displayed chronologically ordered medical data in the first part of the list, and the controller in response to the sensing of the second scroll through action is adapted to control the display unit to display a second part of the list instead of the first part of the list at the second time lying at least partially backward of forward of the previously displayed chronologically ordered medical data in the first part of the list. The controller may further be adapted to control the display unit to display at the same time the first partial area of the first graph and the first part of the list, wherein at least part of the medical data depicted in the first partial area of the first graph are also depicted in the first part of the list.

One or more of the medical data of the first set of medical data and/or the second set of medical data may further be associated with at least one of a plurality of tags. The tags, e.g., provide time related context information for the respective medical data. Non-time related tags, such as tags indicating sports events or illness events may also be used. In one example, the associated tags are also received by the processing unit and the first display signals comprise signals representing the time related tags associated with the respective medical data in the first graph and/or the list, preferably in the list. The plurality of time-related tags comprises, e.g., breakfast, lunch, dinner, bedtime and night tags. A search functionality may be displayed on the display unit for searching the first and second set of medical data for medical data associated with one of the plurality of tags, sensing a search request by the sensing unit for one of the plurality of tags, determining by the controller whether the searched tag is associated with one or more of the medical data of the first and second set of medical data, and providing third display signals to the display unit representing the medical data associated with the searched tag (or a part of the medical data associated with the searched tag) in a second list in chronological order of their times of generation according to the reference concept of time. The third display signals representing the medical data associated with the searched tag (or a part of the medical data associated with the searched tag) in a second list in chronological order of their times of generation according to the reference concept of time are provided only upon the determining step being true that the searched tag is associated with one or more of the medical data of the first and second set of medical data. E.g., the chronologically youngest medical data is displayed at the top of the second list. The second list does not contain any medical data not associated with the searched tag. When only a part of the medical data associated with the searched tag is displayed in the second list, this part comprises the youngest (according to the reference concept of time) medical data of the displayed medical data and optionally one or more additional medical data associated with the searched tag in chronological order of their times of generation according to the reference concept of time. The third display signals may be displayed on the display unit additionally or alternatively to displaying the first display signals. The time of generation according to the reference concept of time is usually not displayed in lists and/or graphs. Instead, next to each medical data of the first set of medical data associated with the searched tag the respective time of generation according to the first local time may be indicated in the second list and next to each medical data of the second set of medical data associated with the searched tag the respective time of generation according to the second local time may be indicated in the second list.

In one embodiment the method comprises displaying a search functionality on the display unit for searching the first and second set of medical data for medical data associated with one of the plurality of tags, sensing a search request by the sensing unit for one of the plurality of tags, determining by the controller whether the searched tag is associated with one or more of the medical data of the first and second set of medical data, and displaying a selected partial area of the first graph on the display unit, the selected partial area comprising at least the chronologically youngest one of the medical data associated with the searched tag. Alternatively, the selected partial area comprises at least one or more medical data of the first set of medical data associated with the searched tag. The selected partial area may be displayed alternatively or additionally to the first graph or a partial area thereof. As another example, the controller is adapted to receive the first display signals and to control the display unit to display a first partial area of the first graph at a first time, a search functionality is displayed on the display unit for searching the first and second set of medical data for medical data associated with one of the plurality of tags, a search request is sensed by the sensing unit for one of the plurality of tags, the controller determines whether the searched tag is associated with one or more of the medical data of the first and second set of medical data, and upon the determining step being true that the searched tag is associated with one or more of the medical data of the first and second set of medical data the controller is adapted to control the display unit to display a selected partial area, the selected partial area of the first graph comprising at least the chronologically youngest one of the medical data associated with the searched tag. The selected partial area can particularly be displayed instead of the first partial area of the first graph at a second time.

Tags add context to medical data. They can help to understand a patient's diabetes and the relationships between certain situations and glucose concentrations, e.g., by finding patterns in the glucose concentrations. Therapy optimization is easier with some context to the data, e.g., in the form of tags.

In a further aspect, a computer program is proposed comprising instructions which, when the program is executed by an electronic disease management system comprising a processing unit and a display unit, specifically a mobile phone such as a smart phone, cause the electronic disease management system to carry out the method as described herein.

The computer program specifically may be designed as an application, e.g., as an App. The App, as an example, may be downloaded onto the mobile phone from a download server.

The computer program may further comprise instructions that, when the program is executed by the mobile phone, further prompt a used to perform or confirm the performance of certain steps.

In a further aspect, a computer-readable storage medium is disclosed, specifically a non-transitory storage medium, comprising instructions which, when executed by an electronic disease management systems comprising a processing unit and a display unit, specifically a mobile phone such as a smart phone, cause the electronic disease management system to carry out the method according to this disclosure.

The computer-readable storage medium may further comprise instructions which, when executed by the mobile device, prompt a user to perform or confirm the performance of one or more steps.

As used herein, the terms “computer-readable data carrier” and “computer-readable storage medium” specifically may refer to non-transitory data storage means, such as a hardware storage medium having stored thereon computer-executable instructions. The computer-readable data carrier or storage medium specifically may be or may comprise a storage medium such as a random-access memory (RAM) and/or a read-only memory (ROM).

The computer program may also be embodied as a computer program product. As used herein, a computer program product may refer to the program as a tradable product. The product may generally exist in an arbitrary format, such as in a paper format, or on a computer-readable data carrier and/or on a computer-readable storage medium. Specifically, the computer program product may be distributed over a data network.

In a further aspect, a mobile device (e.g., a mobile phone) is disclosed. For definitions and options of the mobile device, reference may be made to the description of the method given above or as further outlined below. The mobile device comprises a processing unit and a display unit. The mobile device is configured for performing the method of this disclosure.

As outlined above, the mobile device may comprise at least one processing unit being programmed for performing the method of this disclosure.

Summarizing and without excluding further possible embodiments, the following embodiments may be envisaged:

### EMBODIMENT 1

1. A method for managing medical data by an electronic disease management system (110) comprising a processing unit (102) and a display unit (112), the method comprising:
a) receiving by the processing unit a first set of a patient's medical data (114) wherein the medical data of the first set of medical data are associated with respective first time stamps indicating the respective times of generation of the medical data (116) of the first set of medical data according to a first local time,
b) associating by the processing unit the medical data of the first set of medical data with respective second time stamps indicating the respective times of generation of the medical data of the first set of medical data according to a reference concept of time,
c) receiving by the processing unit a second set of the patient's medical data wherein the medical data of the second set of medical data are associated with respective third time stamps indicating the respective times of generation of the medical data of the second set of medical data according to a second local time,
d) associating by the processing unit the medical data of the second set of medical data with respective fourth time stamps indicating the respective times of generation of the medical data (117) of the second set of medical data according to the reference concept of time,
e) detecting by the processing unit a change of the local time between generation of the first set of medical data and the second set of medical data,
f) providing first display signals by the processing unit representing the first set of medical data and the second set of medical data in a first graph (118) over time wherein the medical data of the first set of medical data and the second set of medical data are in chronological order of their times of generation according to the reference concept of time in the first graph and the spacing between medical data in the direction of time in the first graph is directly proportional to the difference in their times of generation according to the reference concept of time, and the change of the local time (120) between generation of the first set of medical data and the second set of medical data is indicated in the first graph.

2. The method of embodiment 1, wherein the reference concept of time is Coordinated Universal Time.

3. The method of embodiment 1 or 2, wherein:
the medical data of the first set of medical data are analyte concentrations and/or medication doses, and
the medical data of the second set of medical data are further analyte concentrations and/or further medication doses.

4. The method of any one of embodiments 1 to 3, wherein the medical data comprise blood glucose concentrations and/or insulin doses.

5. The method of any one of embodiments 1 to 4, wherein the medical data of the first set of medical data are glucose concentrations, and the medical data of the second set of medical data are further glucose concentrations.

6. The method of any one of embodiments 1 to 5, wherein the method comprises displaying the entire first graph or a partial area thereof at a time on the display unit.

7. The method of any one of embodiments 1 to 6, wherein the method comprises displaying a target zone for the medical data in the first graph.

8. The method of any one of embodiments 1 to 7, comprising:
b1) providing second display signals by the processing unit representing the first set of medical data in a second graph over time wherein the medical data of the first set of medical data are in chronological order of their times of generation according to the first or reference concept of time in the second graph and the spacing between medical data in the direction of time in the second graph is directly proportional to the difference in their times of generation according to the reference concept of time.

9. The method of embodiment 8, wherein the method comprises displaying the entire second graph or a partial area thereof at a time on the display unit.

10. The method of any one of embodiments 8 or 9, wherein the second display signals comprise signals for representing a time scale in the second graph which indicates when the medical data of the first set of medical data were generated according to the first local time.

11. The method of any one of embodiments 1 to 10, wherein the first display signals comprise signals for representing a first time scale part in the first graph which indicates when the medical data of the first set of medical data were generated according to the first local time, and a second time scale part in the first graph which indicates when the medical data of the second set of medical data were generated according to the second local time.

12. The method of any one of embodiments 1 to 11, wherein the second set of medical data is generated and received after the first set of medical data is generated and received.

13. The method of any one of embodiments 1 to 12, wherein the display unit is a touch sensitive display unit comprising:
a controller unit to control the display unit, and
optionally a sensing unit.

14. The method of embodiment 13 wherein the controller is adapted to receive the first display signals and to control the display unit to display the first graph or a partial area thereof at a time.

15. The method of embodiment 13 or 14, wherein the controller is adapted to receive the second set of display signals and to control the display unit to display the second graph or a partial area thereof at a time prior to receiving the second set of medical data.

16. The method of any one of embodiments 1 to 15, wherein the first display signals replace the second display signals.

17. The method of any one of embodiments 1 to 16, wherein the controller is adapted to receive the first display signals and to control the display unit to display a first partial area of the first graph at a first time, wherein the sensing unit is adapted to sense a first scroll through action of the user in backward or forward direction of the displayed chronologically ordered medical data in the first partial area of the first graph, and the controller in response to the sensing of the first scroll through action is adapted to control the display unit to display a second partial area instead of the first partial area of the first graph at a second time, the second partial area of the first graph lying at least partially backward of forward of the previously displayed chronologically ordered medical data in the first partial area of the first graph.

18. The method of any one of embodiments 1 to 17, wherein the second display signals are provided to the display unit when the patient is in the first time zone, and the first display signals are provided to the display unit when the patient is in the second time zone.

19. The method of any one of embodiments 1 to 18, wherein the first display signals comprise additional signals representing the first set of medical data and the second set of medical data in a list (122) in chronological order of their times of generation according to the reference concept of time, wherein next to each medical data of the first set of medical data the respective time of generation according to the first local time is indicated and next to each medical data of the second set of medical data the respective time of generation according to the second local time is indicated.

20. The method of embodiment 19, wherein the method comprises displaying the list or a part thereof on the display unit in addition to displaying the first graph or a partial area of the first graph at a first time.

21. The method of embodiments 19 or 20, wherein the controller is adapted to receive the first display signals and to control the display unit to display a first part of the list on the display unit of the user's electronic device at the first time, wherein the sensing unit is adapted to sense a second scroll through action of the user in backward or forward direction of the displayed chronologically ordered medical data in the first part of the list, and the controller in response to the sensing of the second scroll through action is adapted to control the display unit to display a second part of the list instead of the first part of the list at the second time lying at least partially backward of forward of the previously displayed chronologically ordered medical data in the first part of the list.

22. The method of embodiment 21, wherein the controller is adapted to control the display unit to display at the same time the first partial area of the first graph and the first part of the list, wherein at least part of the medical data depicted in the first partial area of the first graph are also depicted in the first part of the list.

23. The method of any one of embodiments 1 to 22, wherein one or more of the medical data of the first set of medical data and/or the second set of medical data are further associated with at least one of a plurality of tags (124), the tags providing preferably time related context information for the respective medical data, wherein the first display signals comprise signals representing the tags associated with the respective medical data in the first graph and/or the list.

24. The method of embodiments 1 to 23, wherein the plurality of tags comprises breakfast, lunch, dinner, bedtime and night tags.

25. The method of embodiment 23 or 24, the method further comprising:
displaying a search functionality on the display unit for searching the first and second set of medical data for medical data associated with one of the plurality of tags,
sensing a search request by the sensing unit for one of the plurality of tags,
determining by the controller whether the searched tag is associated with one or more of the medical data of the first and second set of medical data, and
providing third display signals to the display unit representing the medical data associated with the searched tag in a second list in chronological order of their times of generation according to the reference concept of time.

26. The method of any one of embodiments 23 to 25, the method further comprising:
displaying a search functionality on the display unit for searching the first and second set of medical data for medical data associated with one of the plurality of tags,
sensing a search request by the sensing unit for one of the plurality of tags,
determining by the controller whether the searched tag is associated with one or more of the medical data of the first and second set of medical data, and
displaying a selected partial area of the first graph on the display unit, the selected partial area comprising at least the chronologically youngest one of the medical data associated with the searched tag.

27. The method of any one of embodiments 23 to 26, wherein next to each medical data of the first set of medical data associated with the searched tag the respective time of generation according to the first local time is indicated and next to each medical data of the second set of medical data associated with the searched tag the respective time of generation according to the second local time is indicated.

28. A computer program comprising instructions which, when the program is executed by an electronic disease management systems comprising a processing unit and a display unit, cause the electronic disease management system to carry out the method according to of any one of embodiments 1 to 27.

29. A mobile device, comprising a processing unit and a display unit, the mobile device being configured for performing the method according to of any one of embodiments 1 to 27.

Further optional features and embodiments will be disclosed in more detail in the subsequent description of embodiments, preferably in conjunction with the dependent claims. Therein, the respective optional features may be realized in an isolated fashion as well as in any arbitrary feasible combination, as the skilled person will realize. The scope of this disclosure is not restricted by the preferred embodiments. The embodiments are schematically depicted in the Figures. Therein, identical reference numbers in these Figures refer to identical or functionally comparable elements.

### BRIEF DESCRIPTION OF THE DRAWINGS

The above-mentioned aspects of exemplary embodiments will become more apparent and will be better understood by reference to the following description of the embodiments taken in conjunction with the accompanying drawings, wherein:

FIG. 1 shows an embodiment of an electronic disease management system;

FIG. 2 shows a flow chart of an embodiment of the method for managing medical data; and

FIG. 3 shows an embodiment of a mobile device.

### DESCRIPTION

The embodiments described below are not intended to be exhaustive or to limit the invention to the precise forms disclosed in the following detailed description. Rather, the embodiments are chosen and described so that others skilled in the art may appreciate and understand the principles and practices of this disclosure.

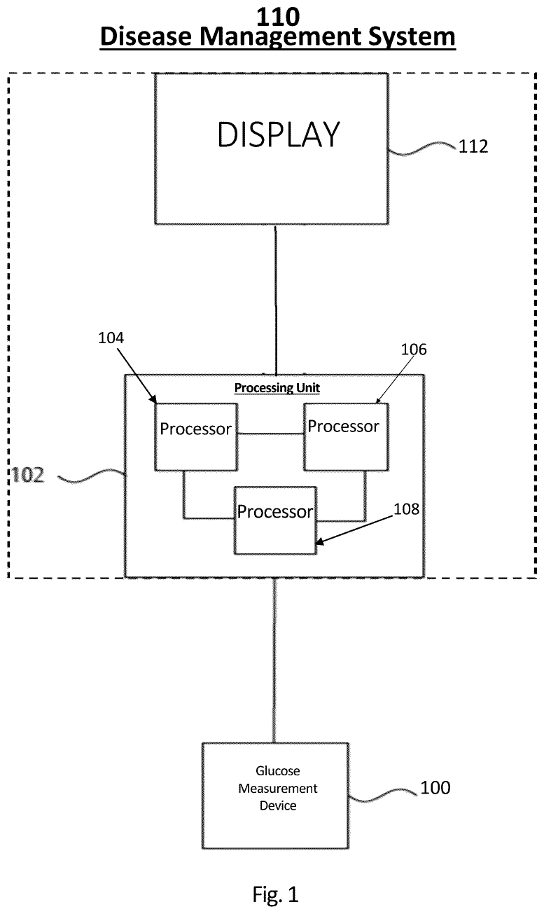

In FIG. 1, an exemplary embodiment of an electronic disease management system 110 is shown. The electronic disease management system comprises a processing unit 102 comprising 3 processors 104, 106 and 108 and a display unit 112. The processing unit 102:
receives a first set of a patient's glucose concentrations associated with respective first time stamps indicating the respective times of generation of the glucose concentrations of the first set of glucose concentrations according to a first local time from a glucose measurement device 100;
associates the glucose concentrations of the first set of glucose concentrations with respective second time stamps indicating the respective times of generation of the glucose concentrations of the first set of glucose concentrations according to a reference concept of time;
receives a second set of a patient's glucose concentrations associated with respective first time stamps indicating the respective times of generation of the glucose concentrations of the second set of glucose concentrations according to a second local time from the glucose measurement device 100;
associates the glucose concentrations of the second set of glucose concentrations with respective fourth time stamps indicating the respective times of generation of the glucose concentrations of the first set of glucose concentrations according to the reference concept of time;
detects a change of the local time between generation of the first set of glucose concentrations and the second set of glucose concentrations; and
provides first display signals to the display unit 112.

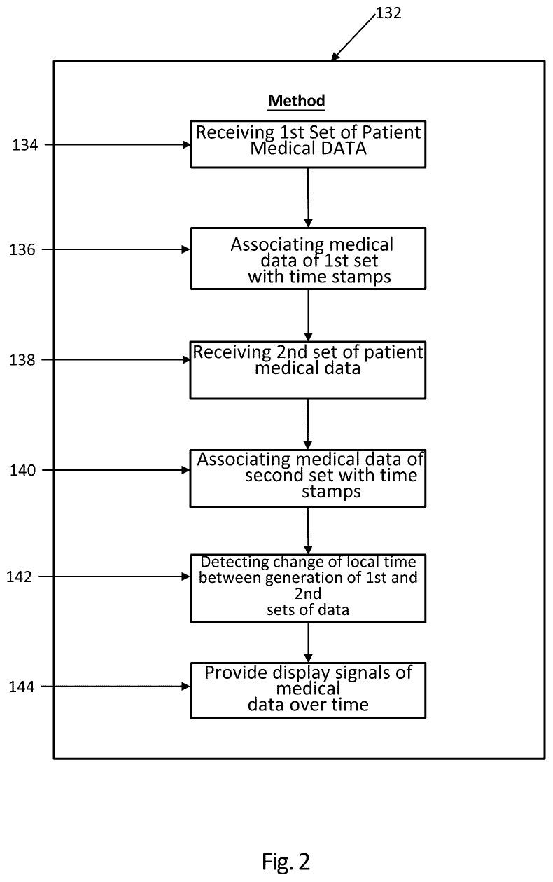

A flow chart of an exemplary embodiment of a method for managing medical data 132 is shown in FIG. 2. The method 132 comprises the following steps, which may specifically be performed in the given order. Still, a different order may also be possible. It may be possible to perform two or more of the method steps fully or partially simultaneously. It may further be possible to perform one, more than one or even all of the method steps once or repeatedly. The method 132 comprises:
a) (denoted with reference number 134) receiving by the processing unit a first set of a patient's medical data (114) wherein the medical data of the first set of medical data are associated with respective first time stamps indicating the respective times of generation of the medical data (116) of the first set of medical data according to a first local time,
b) (denoted with reference number 136) associating by the processing unit the medical data of the first set of medical data with respective second time stamps indicating the respective times of generation of the medical data of the first set of medical data according to a reference concept of time,
c) (denoted with reference number 138) receiving by the processing unit a second set of the patient's medical data wherein the medical data of the second set of medical data are associated with respective third time stamps indicating the respective times of generation of the medical data of the second set of medical data according to a second local time,
d) (denoted with reference number 140) associating by the processing unit the medical data of the second set of medical data with respective fourth time stamps indicating the respective times of generation of the medical data (117) of the second set of medical data according to the reference concept of time,
e) (denoted with reference number 142) detecting by the processing unit a change of the local time between generation of the first set of medical data and the second set of medical data,
f) (denoted with reference number 144) providing first display signals by the processing unit representing the first set of medical data and the second set of medical data in a first graph (118) over time wherein the medical data of the first set of medical data and the second set of medical data are in chronological order of their times of generation according to the reference concept of time in the first graph and the spacing between medical data in the direction of time in the first graph is directly proportional to the difference in their times of generation according to the reference concept of time, and the change of the local time (120) between generation of the first set of medical data and the second set of medical data is indicated in the first graph.

The method 132 may comprise additional method steps that are not listed in FIG. 2.

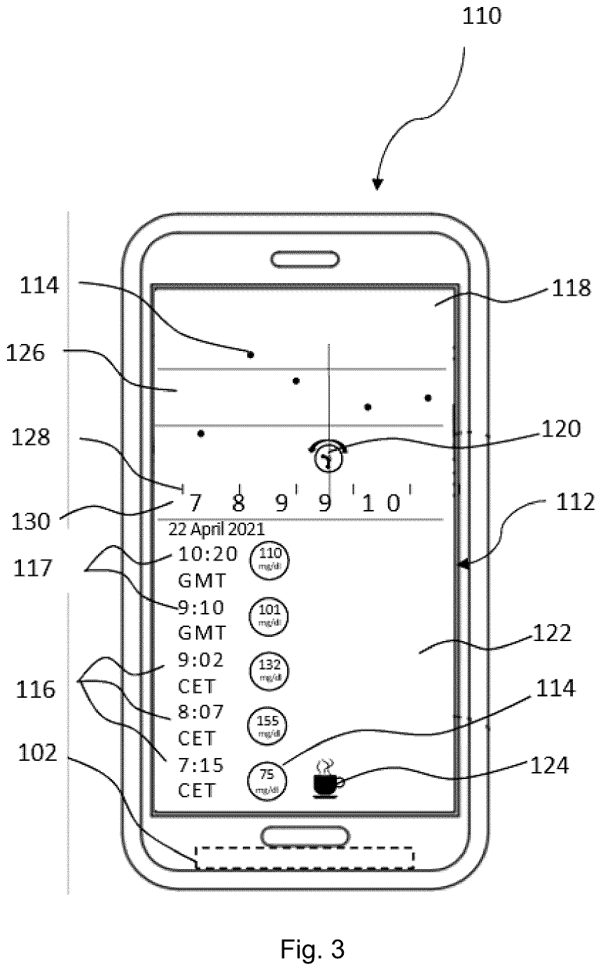

FIG. 3 shows an embodiment of a mobile phone 110 as an example of an electronic disease management system. A front view of the mobile phone is shown. The mobile phone comprises a processing unit 102 located inside the mobile device and therefore is only shown in dashed lines in FIG. 3. The mobile phone further comprises a display unit 112. A first graph 118 over time is displayed in the upper half on the display unit. A list 122 is displayed in the lower half of the display unit. The first graph is a scatter plot to show the change of a patient's glucose concentrations over time. The first graph comprises the first set of glucose concentrations and the second set of glucose concentrations and an indication of the change of the local time 120 between generation of the first set of glucose concentrations and the second set of glucose concentrations. Glucose concentrations 114 are exemplified by black dots in FIG. 3. The three black dots in the left of the first graph 118 represent glucose concentrations of the first set of glucose concentrations patient measured in a first time zone having a first local time, the two black dots in the right of the first graph 118 represent glucose concentrations of the second set of glucose concentrations patient measured in a second time zone having a second local time. The glucose concentrations of the first set and the second set are in chronological order of their times of generation according to the reference concept of time in the first graph and the spacing between the glucose concentrations in the direction of time in the first graph is directly proportional to the difference in their times of generation according to the reference concept of time. A target zone 126 in the first graph indicates the lower and upper boundaries of a glucose target concentration zone for the patient. For example, the boundaries are 80 mg/dl and 140 mg/dl (blood glucose concentration). Blood glucose concentrations below the lower boundary of 80 mg/dl may be considered hypoglycemic events. Blood glucose concentrations above the upper boundary of 140 mg/dl may be considered hyperglycemic events.

A first time scale part is further shown in the first graph which indicates when the glucose concentration of the first set of glucose concentrations were generated according to the first local time (CET as an example), and a second time scale part in the first graph which indicates when the glucose concentrations of the second set of glucose concentrations were generated according to the second local time (GMT as an example). The first time scale part comprises first tick marks (the first of three displayed tick marks being indicated exemplarily as 128) and tick mark time labels (7 o'clock, 8 o'clock, 9 o'clock) (the first of three displayed tick marks time labels being indicated exemplarily as 130) so that the patient can identify based thereon the respective times of generation of the first set of glucose concentrations according to the first local time when looking at the first graph. The second time scale part comprises two second tick marks in the embodiment shown and two second tick mark time labels (9 o'clock, 10 o'clock) so that the patient can identify based thereon the respective times of generation of the second set of glucose concentrations according to the second local time when looking at the first graph. Due to the change of the local time 120 (indicated at about 9:35 o'clock CET) between generation of the first set of medical data and the second set of medical data, there appears to be a non-linearity between the last of the first tick mark labels (9 o'clock) and the first of the second tick mark labels (also 9 o'clock). In the example, the patient travelled, e.g., from Vienna where the first set of glucose concentrations was measured to London (where the second set of glucose concentrations were measured).

The times of generation of the second set of glucose concentrations 117 and the times of generation of the first set of glucose concentrations 116 are shown a in a list (122) in chronological order of their times of generation according to the reference concept of time in the lower part of the display unit. This includes an indication of the respective local time (CET or GMT in the example). The chronologically youngest measured glucose concentrations are displayed at the top of the list with the remaining measured glucose concentrations following in chronological order. The glucose concentrations measured at the indicated times of generation are displayed in circles as an example. As an example 114 indicates a measured glucose concentration of 75 mg/dl (measured at 7:15 CET). This glucose concentration is further associated with a breakfast tag to indicate that the glucose concentrations was measured when starting breakfast. This tag simplifies interpretation of the displayed data by the user. The patient in this case can easily see from the graph that he was in a hypoglycemic state at 7:15 CET when starting breakfast which is an indication that a previously administered night basal insulin dose was not properly chosen.

While exemplary embodiments have been disclosed hereinabove, the present invention is not limited to the disclosed embodiments. Instead, this application is intended to cover any variations, uses, or adaptations of this disclosure using its general principles. Further, this application is intended to cover such departures from the present disclosure as come within known or customary practice in the art to which this invention pertains and which fall within the limits of the appended claims.

### LIST OF REFERENCE NUMBERS

100 glucose measurement device
102 processing unit
104 processor
106 processor
108 processor
110 mobile phone
112 display unit
114 medical data
116 times of generation of medical data of the first set of medical data
117 times of generation of medical data of the second set of medical data
118 first graph
120 change of the local time
122 list
124 tag
126 target zone
128 tick mark
130 tick mark time label
132 method for managing medical data
134 step a)
136 step b)
138 step c)
140 step d)
142 step e)
144 step f)

What is claimed is:

1. A method for managing medical data using an electronic disease management system comprising a processing unit and a display, the method comprising:
a) receiving by the processing unit a first set of a patient's medical data, wherein the medical data of the first set of medical data are associated with respective first time stamps indicating the respective times of generation of the medical data of the first set of medical data according to a first local time;
b) associating by the processing unit the medical data of the first set of medical data with respective second time stamps indicating the respective times of generation of the medical data of the first set of medical data according to a reference concept of time;
c) receiving by the processing unit a second set of the patient's medical data, wherein the medical data of the second set of medical data are associated with respective third time stamps indicating the respective times of generation of the medical data of the second set of medical data according to a second local time;
d) associating by the processing unit the medical data of the second set of medical data with respective fourth time stamps indicating the respective times of generation of the medical data of the second set of medical data according to the reference concept of time;
e) detecting by the processing unit a change of the local time between generation of the first set of medical data and the second set of medical data;
f) providing first display signals by the processing unit representing the first set of medical data and the second set of medical data in a first graph over time wherein the medical data of the first set of medical data and the second set of medical data are in chronological order of their times of generation according to the reference concept of time in the first graph and the spacing between medical data in the direction of time in the first graph is directly proportional to the difference in their times of generation according to the reference concept of time, and the change of the local time between generation of the first set of medical data and the second set of medical data is indicated in the first graph.

2. The method of claim 1, wherein the medical data of the first set of medical data are glucose concentrations, and the medical data of the second set of medical data are further glucose concentrations.

3. The method of claim 1, wherein the method comprises displaying the entire first graph or a partial area thereof at a time on the display.

4. The method of claim 1, wherein the first display signals comprise signals for representing a first time scale part in the first graph which indicates when the medical data of the first set of medical data were generated according to the first local time, and a second time scale part in the first graph which indicates when the medical data of the second set of medical data were generated according to the second local time.

5. The method of claim 1, wherein the display is a touch sensitive display comprising a controller to control the display, and, optionally, a sensing unit, wherein the controller is adapted to receive the first display signals and to control the display to display the first graph or a partial area thereof at a time.

6. The method of claim 5, wherein the controller is adapted to receive the first display signals and to control the display to display a first partial area of the first graph at a first time, wherein the sensing unit is adapted to sense a first scroll through action of the user in backward or forward direction of the displayed chronologically ordered medical data in the first partial area of the first graph, and the controller in response to the sensing of the first scroll through action is adapted to control the display to display a second partial area instead of the first partial area of the first graph at a second time, the second partial area of the first graph lying at least partially backward of forward of the previously displayed chronologically ordered medical data in the first partial area of the first graph.

7. The method of claim 6, wherein the first display signals comprise additional signals representing the first set of medical data and the second set of medical data in a list in chronological order of their times of generation according to the reference concept of time, wherein next to each medical data of the first set of medical data the respective time of generation according to the first local time is indicated and next to each medical data of the second set of medical data the respective time of generation according to the second local time is indicated.

8. The method of claim 7, further comprising displaying the list or a part thereof on the display in addition to displaying the first graph or a partial area of the first graph at a first time.

9. The method of claim 8, wherein the controller is adapted to control the display to display at the same time the first partial area of the first graph and the first part of the list, wherein at least part of the medical data depicted in the first partial area of the first graph are also depicted in the first part of the list.

10. The method of claim 1, wherein one or more of the medical data of the first set of medical data and/or the second set of medical data are further associated with at least one of a plurality of tags, the tags optionally providing time related context information for the respective medical data, wherein the first display signals comprise signals representing the tags associated with the respective medical data in the first graph and/or the list.

11. The method of claim 10, wherein the plurality of tags comprises breakfast, lunch, dinner, bedtime and night tags.

12. The method of claim 10, the method further comprising:
displaying a search functionality on the display for searching the first and second set of medical data for medical data associated with one of the plurality of tags;
sensing a search request by the sensing unit for one of the plurality of tags;
determining by the controller whether the searched tag is associated with one or more of the medical data of the first and second set of medical data; and
providing third display signals to the display representing the medical data associated with the searched tag in a second list in chronological order of their times of generation according to the reference concept of time.

13. The method of claim 10, the method further comprising:
displaying a search functionality on the display for searching the first and second set of medical data for medical data associated with one of the plurality of tags;
sensing a search request by the sensing unit for one of the plurality of tags;
determining by the controller whether the searched tag is associated with one or more of the medical data of the first and second set of medical data; and
displaying a selected partial area of the first graph on the display, the selected partial area comprising at least the chronologically youngest one of the medical data associated with the searched tag.

14. A computer program comprising instructions which, when the program is executed by an electronic disease management systems comprising a processing unit and a display, cause the electronic disease management system to carry out the method according to claim 1.

15. A mobile device, comprising a processing unit and a display, the mobile device being configured for performing the method according to claim 1.
